(19) United States
(12) Patent Application Publication
(10) Pub. No.: US 2007/0054416 A1
(43) Pub. Date: Mar. 8, 2007

(54) High density sample holder for analysis of biological samples

(76) Inventors: Fred E. Regnier, West Lafayette, IN (US); Noubar B. Afeyan, Lexington, MA (US); Marvin Vestal, Framingham, MA (US)

Correspondence Address: KIRKPATRICK & LOCKHART NICHOLSON GRAHAM LLP, One Lincoln Street, BOSTON, MA, 02111-2950, US

(21) Appl. No.: 11/593,223

(22) Filed: Nov. 6, 2006

Related U.S. Application Data
(63) Continuation of application No. 10/117,453, filed on Apr. 5, 2002.
Application No. 10/117,453 is a continuation of application No. 09/102,934, filed on Jun. 23, 1998, now abandoned.
(60) Provisional application No. 60/050,840, filed on Jun. 26, 1997.

(51) Int. Cl. G01N 33/543; C12M 1/34

(52) U.S. Cl. 436/518; 436/86; 435/287.2

(57) ABSTRACT

A sample holder comprises a substrate microfabricated to define a multiplicity of microscopic islands defining sample support surfaces. At least one sump separates adjacent island surfaces and inhibits transport of samples between adjacent island surfaces.

### RELATED APPLICATION

This application claims priority to provisional application U.S.S.N. 60/050,840 filed on Jun. 26, 1997.

### FIELD OF THE INVENTION

The invention relates to a sample holder for chemical analysis and synthesis of samples and more specifically to a high density sample holder for holding and maintaining separation of multiple samples during a chemical analysis or synthesis process.

### BACKGROUND OF THE INVENTION

Molecular biology comprises a wide variety of techniques for the analysis of biological samples including nucleic acids and proteins, many of which form the basis of clinical diagnostic assays. These techniques, for example, include nucleic acid hybridization analysis, restriction enzyme analysis, genetic sequence analysis, ligand/receptor binding assays, and separation and purification of nucleic acids and proteins. Many investigatory molecular biology techniques involve carrying out numerous operations on a large number of samples. These operations are often complex and time consuming and generally require a high degree of accuracy.

Matrix-assisted laser desorption ionization (MALDI) is a technique that allows very large molecules, such as DNA fragments and proteins, to be desorbed from a solid sample and ionized without significant decomposition. Coupled with mass spectrometry, the MALDI technique allows the molecular weights of biological polymers and other large molecules, including industrial polymers, to be precisely determined.

Typically, in a MALDI process, a sample plate contains one or a plurality of small aliquots of a mixture of the sample to be analyzed and an appropriate matrix. After the samples on the sample plate are dried, the sample plate is placed inside a vacuum chamber. A laser beam strikes each sample to desorb and ionize sample molecules, thereby creating an ion cloud for each sample. Ions in the cloud are extracted by electrical fields and travel toward a detector. Ions of different masses require different times to travel toward the detector. Therefore, by measuring the time it takes for an ion beam to reach the detector, one can determine the molecular weight of the sample. The laser beam strikes one sample spot at a time typically by moving the sample plate relative to the laser beam.

In general, a sample plate used in a MALDI process is a substrate having a flat surface. A conventional sample plate has several drawbacks. The conventional sample plate may allow remixing of previously separated samples. When the samples are first placed on the sample plate, the samples may be physically separated and distinguishable. However, after the sample solution is deposited on the sample plate, the samples may spread by diffusion and become mixed with other samples, because there is no barrier between the samples. To avoid sample diffusion and mixing, samples may be sufficiently spaced apart. When the samples are spaced apart, however, sample density on the sample plate is reduced, and thus multiple plates may be required to analyze a large number of samples. Since only one sample plate is placed in a MALDI chamber at a time, and since it takes a while to create the necessary vacuum in the chamber, rapid analysis of a large number of samples becomes difficult.

Another problem with the conventional sample plate is that samples are wasted. A sample droplet placed on the sample plate typically comprises a relatively large surface area as compared to the laser beam diameter. The sample droplet can cover about 2 mm², and a laser beam diameter typically is approximately 100 μm². Thus the laser beam strikes only a tiny fraction of the sample, wasting the rest of the sample. Yet another problem with the conventional sample plate is that when multiple samples are placed on the plate, it is difficult to address each sample on the plate. The ability to address each sample is important as samples that are individually addressable can be selectively analyzed. Still another problem with the conventional sample plate is that the analysis can be slow, because the sample may not uniformly cover the surface of the sample plate. In preparing samples for MALDI a small amount of sample typically is mixed with a large amount of matrix liquid. The mixture is placed on the sample plate and allowed to spread and dry on the sample plate surface. Since the mixture comprises a small amount of the sample, certain areas of the plate surface may not have any sample at all when the sample dries. Therefore, during a MALDI analysis, the laser beam must strike multiple spots in order to gather enough data from spots supporting the sample. In general, a MALDI analysis for a sample takes a minimum of 30 seconds.

U.S. Pat. No. 5,498,545 describes a sample plate that comprises physically separated sample spots. The sample spots are separated either by etching multiple holes in a substrate as shown in FIG. 1, or by placing pins on a substrate. A spot diameter is in the millimeter range. These spots are macroscopic in area and are much larger than the area of the laser beam.

Conventional sample plates and probes used in mass spectroscopy analysis in biological screening, as a holder for a library of biopolymers, or as a plate for biological synthesis or analysis also have flat surfaces, macroscopic sample spots, or wells. Therefore, some of the problems discussed with respect to MALDI sample plates such as sample diffusion, small sample density, and wasted samples also exist with these plates.

### SUMMARY OF THE INVENTION

In one aspect, the present invention provides a sample holder that overcomes many of the problems of convention sample holders. The present sample holder supports a multiplicity of samples on a small space, while permitting physical separation of these samples. The present sample holder furthers allows each sample location to be addressable such that selective analysis or synthesis of a sample can be performed.

The sample holder comprises a substrate microfabricated to define a multiplicity of microscopic islands separated by at least one sump. The microscopic islands define sample support surfaces and the at least one sump inhibits transport of samples between adjacent sample support surfaces. In one embodiment, the at least one sump is a plurality of interconnected sumps forming a gutter, and each sump comprises at least one hole for drainage. In another embodiment, the plurality of interconnected sumps are disposed on a first surface of the substrate and the sample holder further comprises a second group of interconnected sumps disposed on the second surface of the substrate. The second group of interconnected sumps are offset from the first group of interconnected sumps such that a multiplicity of vertical holes that extend through the thickness of the substrate form where the first group of interconnected sumps intersect with the second group of interconnected sumps. In yet another embodiment, the multiplicity of islands comprises sub-islands separated by at least one sub-sump disposed on the sample support surface. In still another embodiment, the sample holder is disposed within the sample chamber of a matrix assisted laser desorption ionization mass spectrometer.

In another aspect, the invention features a system for rapid analysis of a plurality of samples. The system comprises a vacuum controllable chamber, a sample holder for disposition within the chamber for holding a plurality of samples, a laser source and a mass spectrometer. The sample holder comprises a substrate microfabricated to define a multiplicity of microscopic islands separated by at least one sump. The islands define sample support surfaces and the at least one sump inhibits transport of samples between adjacent surfaces. The laser source has means for generating and directing a laser beam for striking a sample on a sample support surface to desorb and ionize sample molecules therefrom. The mass spectrometer analyzes samples on the sample holder by detecting the mass thereof In one embodiment, the optical means is the means for directing a laser beam. In another embodiment, the system further includes a mechanism for moving the sample holder relative to the laser beam such that each surface of the sample support surfaces is impinged by a stationary laser beam. In still another embodiment, each sample support surface has a surface area that is approximately equal to or smaller than a diameter of the laser beam striking each sample.

In still another aspect, the invention features a method for analyzing a biological sample. According to the method, a sample holder comprising a substrate microfabricated to define a multiplicity of microscopic islands defining sample support surfaces is provided. At least one sump, separates adjacent support surfaces and inhibits transport of samples between adjacent surfaces. A plurality of samples are placed in contact with the sample support surfaces, and an analysis step is performed on the biological sample. In one embodiment, the analysis step comprises detection by matrix assisted laser desorption ionization mass spectrometry. In another embodiment, the analysis step comprises immunoassay. In still another embodiment, the method further includes the step of fabricating a library of polymers on the sample holder such that each polymer is disposed on a sample support surface. The library of polymers may be a library of biopolymers such as peptides, oligonucleotides, or probes for biological screening.

### BRIEF DESCRIPTION OF THE DRAWINGS

These and other advantages of the invention may be more clearly understood with reference to the specification and the drawings, in which:

FIG. 1 shows a cross-section view of a prior art sample holder.

FIG. 2A shows a perspective view of an embodiment of the sample holder of the present invention.

FIG. 2B shows a top view of a section of the sample holder of FIG. 2A.

FIG. 2C shows a cross-section view of a section of the sample holder of FIG. 2A cut through 2C′-2C″.

FIG. 3A shows a top view of a section of an embodiment of the sample holder of the prevent invention.

FIG. 3B shows a cross-section view of the sample holder of FIG. 3A cut through 3B′-B″.

FIG. 3C shows a cross-section view of the sample holder of FIG. 3A cut through 3C′-C″.

FIG. 4 illustrates inhibition of sample transport to adjacent sample support surfaces using the sample holder of FIG. C.

FIG. 5 shows a top view of an embodiment of the sample holder of the present invention.

FIG. 6A shows a top view of a section of an embodiment of the sample holder of the present invention.

FIG. 6B shows a cross-section view of the sample holder of FIG. 6A cut through 6B′-6B″.

FIG. 7 shows a rear surface of the sample holder of FIG. 2B.

FIG. 8 shows a schematic diagram of a MALDI chamber.

FIG. 9 illustrates a method of transferring samples onto the sample holder of the present invention.

FIG. 10 shows a perspective view of an embodiment of the sample holder of the present invention.

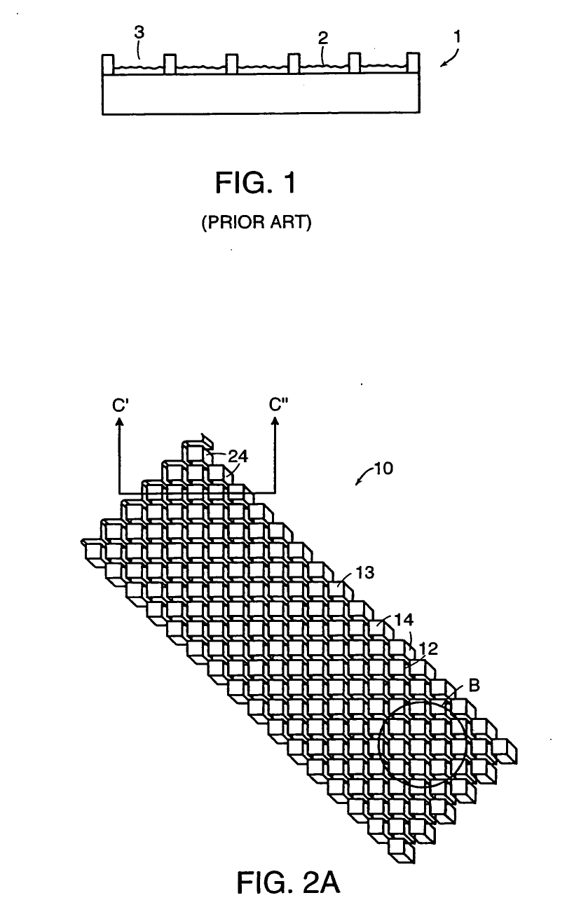

### DETAILED DESCRIPTION OF THE INVENTION

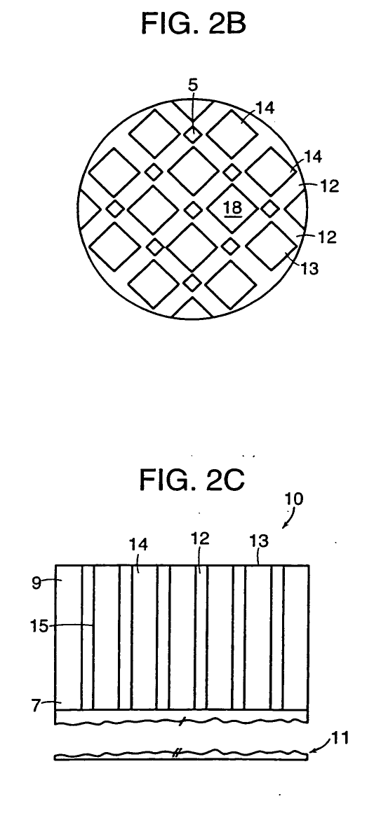

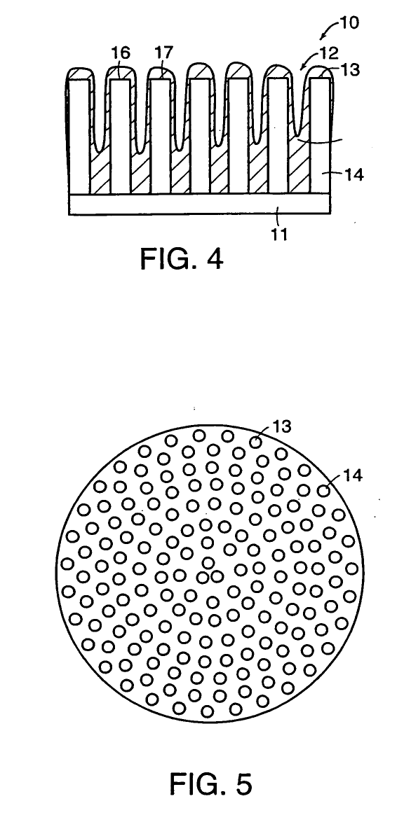

Referring to FIGS. 2A, 2B, 2C, a sample holder 10, constructed in accordance with the invention comprises a substrate 11 microfabricated to define a multiplicity of microscopic islands 14 defining upper sample support surfaces 13. The term “island” refers to a structure protruding from the substrate 11. A sample support surface 13 is an exposed surface on an island that typically is substantially parallel to the horizontal plane of the substrate 11, unlike prior art sample holders 1, which hold the samples 2 in the wells 3. At least one sump 12 separates the surfaces 13 of adjacent islands 14. The term “sump” refers to a recessed area between adjacent islands 14. The sump 12 is recessed below the surfaces 13 of the adjacent islands 14. In a preferred embodiment, the sump 12 is recessed by at least 10 microns. As illustrated in FIG. 4, the sump 12 inhibits transport of samples between the surfaces 13 of the islands 14 by collecting the sample that transports (e.g., diffuses) away from one surface 16 towards an adjacent island surface 17. Thus excess sample applied to a surface 13 drains into the sump 12 and is less likely to mix with sample on an adjacent surface. In general, a sample placed on a portion of a surface tends to diffuse to neighboring portions of the surface over time. Referring to FIG. 2C, the islands 14 connect to one another at a first end 7 by the substrate 11. However, the remainder of each island 14 is physically separated from the adjacent islands 14. Each island 14 has at least one exposed surface 13 for supporting a sample.

In one embodiment, the sumps 12 comprise at least one hole 5 for draining the samples that collect in the sumps 12 away from the sample holder 10 as shown in FIG. 2B. For example, a vacuum may be applied to the back of the sample holder 10 to suck the material collecting in the sumps 12 out of the sumps 12. In a preferred embodiment, the sumps 12 in the sample holder 10 are interconnected to form a gutter as shown in FIGS. 2A, 2B, and 2C. A gutter better inhibits the samples from transporting towards adjacent island surfaces 13 by draining the samples that transport away from the sample support surfaces 13. The gutter is wide enough to minimize surface tension between the samples and the gutter surfaces to induce the samples to flow down to the bottom of the sumps 12 and to induce flow of the samples in the gutter for drainage.

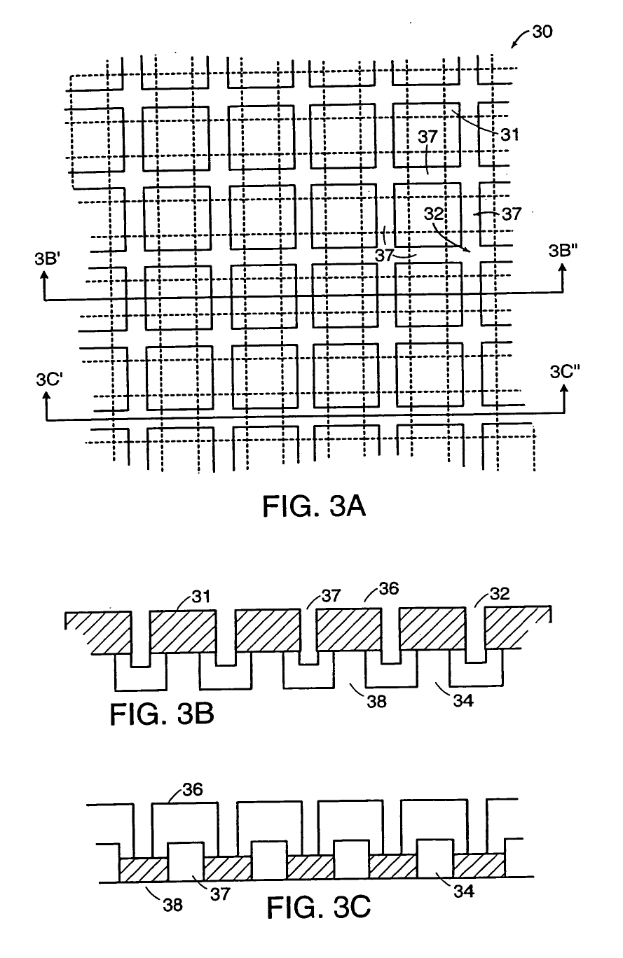

Referring to FIGS. 3A, 3B and 3C, in a preferred embodiment, the sample holder 30 drains the samples collected in the sumps 32. The sample holder 30 includes a first group of interconnected sumps 32 forming a first gutter and a second group of interconnected sumps 34 forming a second gutter. The first group of interconnected sumps 32 and the sample support surfaces 31 are disposed on the first surface of the substrate 36 and the second group of interconnected sumps 34 are disposed on the second surface of the substrate 38. The second group of interconnected sumps 34 are disposed offset from the first group of interconnected sumps 32 such that the first group of interconnected sumps 32 intersecting the second group of interconnected sumps 34 create a plurality of holes 37 that are substantially perpendicular to the substrate and that extend through the thickness of the substrate. In the embodiment shown in FIGS. 3A, 3B, and 3C, each sample support surface 31 is surrounded by four holes 37 for draining the samples away from the sample holder 30. The size and shape of the first group of interconnected sumps 32 may, but need not be the same as the size and shape of the second group of interconnected sumps 34.

In another embodiment, the sample holder comprises a substrate defining a plurality of sample support surfaces. Each sample support surface is surrounded by a plurality of vertical channels that extend through the substrate, such that excess sample placed on a sample support surface drains away from the sample surface through the channels.

Referring to FIG. 10, in still another embodiment, the sample holder 80 comprises a first substrate 82 and a second substrate 84 attached to the first substrate 82. The first substrate 82 comprises a plurality of sample support surfaces 86 and a plurality of vertical channels 88 adjacent to the sample support surfaces 86. The vertical channels 88 extend through the thickness of the first substrate 82. The second substrate 84 comprises a plurality of sumps 90 or interconnected sumps defining. a gutter. The plurality of sumps 90 of the second substrate 84 are in communication with the plurality of vertical channels 88 of the first substrate 82.

In the embodiments of FIG. 2A, 2B, and 2C, the islands 14 are substantially identical in size and shape and the sumps 12 also are substantially identical in size and shape. Uniformity in the size or shape of the islands 14, the sample support surfaces 13, or the sumps 12, although acceptable, is not a requirement of the present invention. In one exemplary embodiment, the sample support surface 13 areas on a sample holder 10 vary depending on a characteristic of a sample placed on each surface 13.

Each island 14 has a sample support surface area 13 that is microscopic in size. The term “microscopic” refers to an area less than about 10,000 μm², preferably less than about 5,000 μm², more preferably less than about 1000 μm² and in many embodiments less than 100 μm ². Smaller sample support surface 13 areas along with narrow distances between adjacent islands 14 provide the advantage of increasing sample density on a sample holder 10. In one embodiment, the sample holder 10 comprises more than 1000 islands per cm² of substrate area 11. In a preferred embodiment, the sample holder 10 comprises more than 10,000 islands per cm² of substrate area. The selected height of an island 14 (or depth of the sump 12) depends in part on the intended sample volume to be placed on the sample support surface 13 of the island 14. The term “height” refers to the distance from a first end 7 to a second end 9 of an island 14. The height of an island 14 preferably is large enough to prevent the sample on the sample support surface 13 from transporting and reaching the adjacent island surfaces 13. The height of an island 14 and the distance between the island and an adjacent island 14 define the size of the sump 12 between the two islands. In one embodiment, the distance between adjacent islands 14 is less than approximately 100 μm. In preferred embodiments, the distance between adjacent islands is less than approximately 50 μm, but great enough to prevent a viscous liquid from bridging the channel between adjacent islands. In a preferred embodiment, the sumps 12 are deep and narrow as permissible by the state of fabrication. Existing fabrication technologies permit creation of a sump 12 having a depth that is 20 times greater than its width. For example, a sump 12 having a width of 10 microns may have a depth of up to 200 microns. Since dimensions of a sump 12 is limited by existing fabrication technologies, a greater depth along with a narrower width is expected with advances in such technologies.

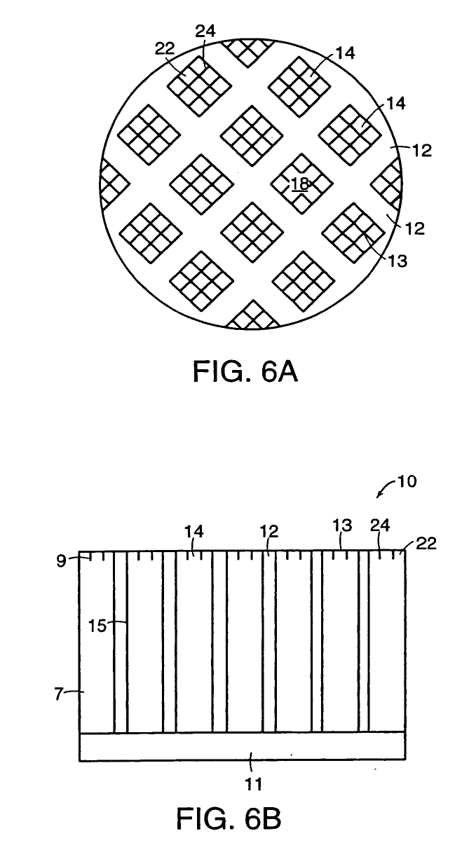

The islands 14 and the sample support surfaces 13 are individually addressable, for example, with a laser beam, according to the present invention. The sample holder 10 may have at least two or more reference points to assist in identification of sample locations. Furthermore, an island 14 or a sample support surface 13 is repeatedly addressable such that multiple analysis or synthesis can be performed on a sample disposed on a particular sample support surface 13 or a collection of adjacent surfaces. In one embodiment, the islands 14 on the sample holder 10 form an array. In the embodiment of FIGS. 2A, 2B and 2C, the islands 14 form a two dimensional array and the sample support surfaces 13 are substantially square. According to the present invention, the sample support surfaces 13 may be of any shape sufficient to support a sample. In the embodiment of FIG. 5, the islands 14 form concentric islands 14, forming sample support surfaces 13 that are ring-shaped. This permits address of a particular surface by rotation of the sample holder 10 and radial movement of a device for use in approaching a particular surface. In the embodiment of FIGS. 6A and 6B, the islands 14 form sub-islands 22 on each sample support surface 13. The sub-islands 22 are separated by at least one sub-sump 24 disposed on the sample support surface 13. The configuration including sub-islands 22 permits placement of multiple samples on a sample support surface 13. The sub-islands 20 may form an array.

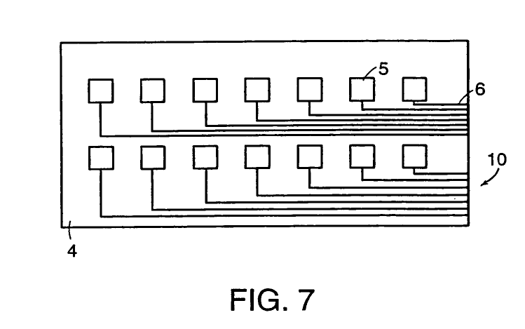

In another embodiment, voltage can be directed to specific sample support surfaces of the sample holder 10. Referring to FIG. 7, a sample holder 10 has a plurality of drain holes 5 that extend though the thickness of the sample holder 10. Each drain hole 5 is disposed in a sump in between two or more adjacent islands. Metal leads 6 are microfabricated on a rear side 4 of the sample holder 10, and each lead 6 extends to a drain hole 5. At least some of the sample support surfaces and the walls of the sumps are metallized, such that votage can be directed to these support surfaces.

A variety of techniques can create the islands 14 and the sumps 12. In one embodiment, the islands 14 and the sumps 12 are created by etching the sumps 12. Both isotropic and anisotropic etching methods can be used to create the sumps 12, but anisotropic etching technique is preferred because anisotropic etching techniques are capable of creating deep, vertical, narrow channels. Anisotropic etching techniques, for example, include deep reactive ion etching, electron beam etching and LIGA (Lithographie Galvanoformung Abformung). These etching techniques are well known in the art. LIGA is a process that allows fabrication of three dimensional structures having high aspect ratios. The process involves four steps: irradiation, development, electroforming and resist stripping. Irradiation step involves irradiating a resist using laser, electron-beam or X-ray from a synchrotron radiation source. In the development step, a pattern is transferred into the resist and the resist is etched to reveal three dimensional structures comprising the resist material. In the electroforming step, a metallic mold is produced around the resist structures by electroplating. In the final step, the resist is stripped to reveal channels. Anisotropic wet etching may also be used to create the sumps 12. Anisotropic wet etching, however, requires a specific type of substrate 11. For example, the substrate 11 must be crystalline and etching occurs along a specific axis.

In fabricating the sample holder 10 by an etching process, a substrate 11 is first provided. In one embodiment, the substrate 11 comprises a conductive material. A conductive substrate, or one permitting flow of charge to or from the sample, for example, is suitable for fabricating a MALDI sample holder. A substrate can be made conductive by coating an inorganic or organic substrate with a conductive material. For example, gold may be sputtered onto a nonconductive substrate. Alternatively, the substrate 11 may comprise a metal, a glass, a plastic or any other material suitable for supporting a sample. The substrate 11 is patterned to designate areas to be etched. The patterned substrate 11 is etched to create the islands 14 and the sumps 12. For the embodiments in which the sample holder 30 comprises a first and a second group of interconnected sumps 32, 34 as shown in FIGS. 3A, 3B and 3C, both a first and a second surface of the substrate 11 are patterned and etched. For the embodiments in which the sumps 12 comprise at least one hole 5 for draining as shown in FIG. 2B, an additional etching step may be performed to create the holes 5. For example, holes having dimensions of approximately 10 microns by 10 microns may be etched through the thickness of a substrate having a thickness in the range of from about 25 microns to about 100 microns, using LIGA or deep reactive ion etching techniques.

In another embodiment, the islands 14 are grown on the substrate 11 using a microfabrication deposition technology well known in the art. Alternately, the islands 14 are created using a technique referred to as the “poor man's LIGA”. According to this technique, a 30-50 micron thick layer of photoresist is placed on a substrate 11, patterned as in a conventional lithography to define islands 14 and sumps 12, and washed with a solvent. Areas in which the photoresists has been washed away define sumps 12 and remaining photoresist structures define islands14. In still another embodiment, the islands 14 may be fabricated independently and subsequently bonded to the substrate 11.

In one embodiment, the sample support surfaces 13 include a surface coating designed, for example, to enhance sample adhesion to the support surfaces 13 to provide for selective adsorption of samples in various regions of the holder, or to change surface properties such as wetting properties of the surfaces 13. For example, cationic or anionic moities, chelators, organic molecules including complex sugars and heparin, binding proteins such as antibodies, avidin, etc., hydrophobic coatings (e.g., octadecyl silane) may enhance sample adhesion or selectivity on the sample support surfaces 13. These may take the form of adhered coating material bonded or simply adsorbed onto surfaces 13 and may be in any form including gels, fimbriae and arborial coatings. The coating layer may be as thin as a few angstroms and as thick as desired. Any coating technology known (or hereinafter discovered) by those skilled in the art may be used to coat the sample support surfaces 13. The particular coating technology exploited for coating the sample support surfaces 13 does not constitute an aspect of the present invention as currently understood.

In another embodiment, the surfaces comprise a MALDI matrix of a type known per se, ready to receive a sample for analysis in a MALDI mass spectrometer.

In one embodiment, the sample support surfaces 13 are smooth. In another embodiment, the sample support surfaces 13 are irregular. An irregular surface increases the surface area, and thereby increases the amount of sample placed on the surface. Irregular surfaces also enhance adhesion of the sample onto the sample support surfaces 13 by improving physical bonding between the samples and the surfaces 13. A variety of methods can make the sample support surfaces 13 irregular. In one exemplary embodiment, multiple etching steps may be performed to create sumps having varying depth. The first etching step may create sub-sumps that are 10 micron deep and the second etching step may create sumps that are 100 microns deep, such that each island 14 comprises a plurality of sub-sumps making the sample support surface on the island 14 irregular. In another exemplary embodiment, an organic coating such as fimbriated organic coating is applied on the sample support surfaces 13. A fimbriated organic coating may increase the loading capacity by three to five times. In still another exemplary embodiment, the sample support surfaces 13 are etched in an anodization process to create pores that have depth of 1-2 microns, diameter of 10-500 angstroms, porosity of 50-70% and surface area of greater than 100 m²/g. The anodization process is described in L. T. Canham, “Bioactive Silicon Structure Fabrication Through Nanoetching Techniques”, Advanced Materials, 7:2033 (1995).

In one embodiment, the gutter or sump 12 surfaces may be treated to modify the wetting properties of these surfaces. In order to improve wettability and thereby allow samples to spread and collect at the bottom of the sumps 12, the sump surfaces may be coated, for example, with a surfactant or a hydrophobic substance. Any surface treatment technology known (or hereinafter discovered) by those skilled in the art may be used to modify the wetting properties of the sump surfaces. The particular method of modifying wetting properties of the sump surfaces does not constitute an aspect of the present invention.

The sample holder 10 of the present invention may be used to support a large number of densely placed samples for use in a chemical analysis or a biological synthesis.

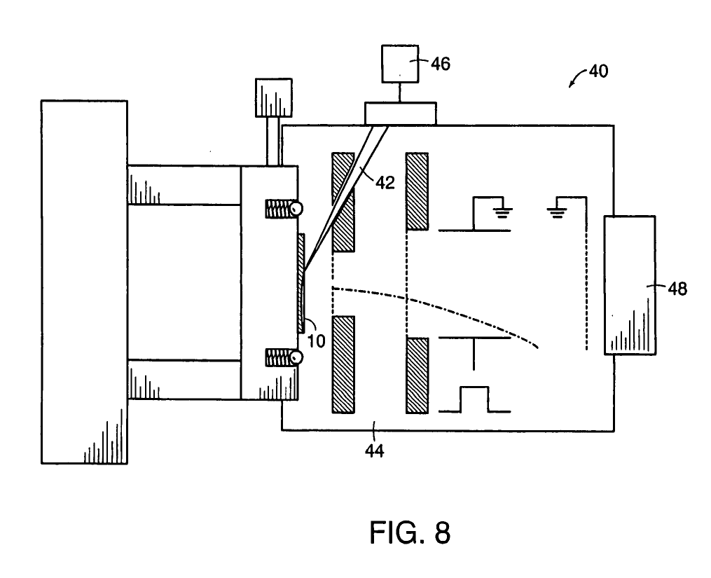

Referring to FIG. 8, the sample holder 10 of the present invention is used as a sample plate in a MALDI apparatus 40. A MALDI apparatus 40 comprises a vacuum chamber 44, a sample holder 10 placed inside the chamber 44 for holding a multiplicity of samples, a laser source 46, and a mass spectrometer 48. The laser source 46 generates a laser beam 42 for striking an addressed sample on a selected sample support surface 13 to desorb and ionize sample molecules disposed therein. The mass spectrometer 48 analyzes serially the multiplicity of samples by detecting the desorbed and ionized sample molecules at a corresponding multiplicity of islands. The sample holder 10 allows placement of a large number of samples that are physically separated from each other on a small surface. For example, a sample holder 10 having a dimension of 2 inches by 2 inches (4,4×4.4 cm) may support as many as 250,000 samples (or more), each sample being effectively isolated on a sample support surface 13 of an island 14 or a group of such support surfaces. The dimensions chosen for the sample support surface 13 can depend in part on the diameter of the laser beam 42 impinging on a sample. In one embodiment, each sample support surface 13 has an area approximately equal to or slightly less than the area of the laser beam 42, such that substantially all of the sample placed on the support structure 14 is illuminated by the laser beam 42 when the address is accurately specified. The diameter of a typical laser beam 42 currently used in MALDI analysis is approximately 100 μm². However, it is possible to reduce the laser beam diameter to about 5 μm². Alternatively, a small area laser beam may be used to desorb and ionize species on an addressed island in multiple locations. In another embodiment, a sample support surface 13 is fabricated to have a small surface in order to concentrate a sample on the sample support surface 13. During a drying stage of sample preparation, the sample can spread to wherever there is the liquid matrix. Therefore, on a larger surface, the sample can spread to a wider area creating a less concentrated sample, while on a smaller surface, the sample spreads to a narrower area creating a more concentrated sample. Concentrated sample can lead to MALDI spectra with better resolution.

In addition, the sample holder 10 allows identification of each sample location on the sample holder 10, such that each sample is selectively addressable. In this manner, MALDI analysis can be performed selectively on a desired sample. The sample position relative to the laser beam 42 can be adjusted in a variety of ways. In one embodiment, the sample holder 10 is placed on an x-y table and the x-y position is controlled by one or more stepper motors conventionally used with x-y tables. With computer control of the stepper motors, this system allows any selected point on the sample holder 10 to be positioned precisely on the optical path of the laser beam 42. In another embodiment, conventional optics are used to alter the direction of the laser beam 42 relative to the sample holder 10, thereby allowing different sample support surfaces 13 to be irradiated. Methods for varying sample position or laser beam position are well known in the art and are not aspects of the present invention.

The depth, width, and length of the sumps 12 depend on numerous factors including desired sample density on the sample holder 10, sample volume placed on each sample support surface 13, and minimum incremental distance at which the laser beam can be maneuvered relative to the sample support surfaces 13. A narrower sump 12 (or shorter distance between adjacent islands 14) allows for a greater number of islands 14 to be created on a sample holder 10. However, a shorter distance between islands 14 also results in a smaller sump volume, unless the sump 12 can be made deep. A short distance between islands 14 may also allow viscous liquids to bridge the channel between adjacent island 14. Current microfabrication technologies allow creation of channels having an aspect ratio of up to about 100. An aspect ratio refers to ratio of a depth to a width of a channel, where the depth is a dimension perpendicular to a substrate and the width is a shortest dimension parallel to the substrate. Current typical microfabrication technology allows resolution in the production of masks and etching to about 0.1 μm. Therefore, the lower limitation on a channel width is approximately 0.5±0.1 μm. Since, sump dimensions are limited by fabrication limitations and not design, sumps 12 having greater aspect ratios are expected as fabrication technologies advance. In embodiments where the sumps 12 have drain holes 5, efficient removal of the transported samples preclude the need for deeper and wider sumps. However, in embodiments having sumps 12 without the drain holes 5, the sumps 12 must be large enough and deep enough to keep diffused samples from reaching other samples placed on adjacent sample support surfaces 13.

MALDI analysis using the sample holder 10 of the present invention provide rapid analysis of a large number of samples by allowing a multiplicity of samples to be placed on a small surface without diffusing into each other. In one embodiment, the analysis for a sample takes less than approximately a second, where each analysis comprising impinging the sample with greater than approximately 10 pulses of laser beams. In another embodiment, the analysis for a sample takes less than approximately 100 msec, where each analysis comprises impinging the sample with approximately 100 pulses of laser beams.

Physically separated distinguishable samples may be loaded on the sample holder 10 using a variety of methods. In one embodiment, the samples separated using two dimensional gel electrophoresis, are loaded onto the sample holder 10 using electroblotting or elution.

Two dimensional gel electrophoresis involves two sequential separations performed orthogonally in a gel media typically exploiting two distinct separation criteria, e.g. isoelectric focusing followed by gel electrophoresis. Isoelectric focusing separates proteins according to charge and gel electrophoresis separates proteins using molecular size. The two dimensional gel electrophoresis creates a two dimensional pattern of spots, each spot typically consisting of a specific protein. Two-dimensional electrophoresis is well known in the art.

Electroblotting involves transfer of proteins from the gel onto another surface using an electric current to drive their migration in a manner similar to the original electrophoresis, but in a perpendicular direction. Electroblotting is well known in the art. Standard dot blotting also may be used if the surfaces 13 of the holder are treated to adsorb or absorb a sample.

For the electroblotting embodiment, the sample holder 10 may comprise a multiplicity of electrodes, microfabricated during manufacture of the holder 10 using conventional solid state circuit microfabrication techniques, which permit one to selectively electroblot a biological sample that is pre-separated into a two dimensional field, e.g. by 2D electrophoresis. Each surface 13 or a subset thereof, may be designed to be addressed individually so as to electrically attract sample, or not, as desired. The proteins or other molecules are selectively electroblotted onto the sample support surfaces 13, such that each is disposed on a sample support surface 13.

In one electroblotting embodiment, the sample support surfaces 13 of the sample holder 10 may comprise a sputtered gold layer, which performs as the electrodes, and a hydrophobic coating layer comprising a mixture of CH₃(CH₂)₇SH and HS—CH₂CO₂H, disposed above the gold layer. The mixture provides holes in the hydrophobic coating such that current may reach the gold layer surface. Blotting occurs by applying voltage to the gold layer. In a preferred embodiment, the blotting takes placed in a chamber under Helium pressure. This environment dissolves gas created by electrolysis at the gold surface, which would otherwise impede electrophoresis. In another blotting embodiment, the sample holder 10 comprises a silica plate coated with C₁₈. The silica plate has holes that extend through the thickness of the plate, located approximately every 100 microns. The gel comprising separated proteins is disposed over the silica plate, and a voltage is applied across the rear surface of the sample holder 10 and the gel. The sample 10 holder has metal leads microfabricated on the rear surface. As proteins travel laterally to reach the holes, they contact the sample support surfaces 13 and adsorb thereto. A completely flat surface would create circles of analytes around the holes.

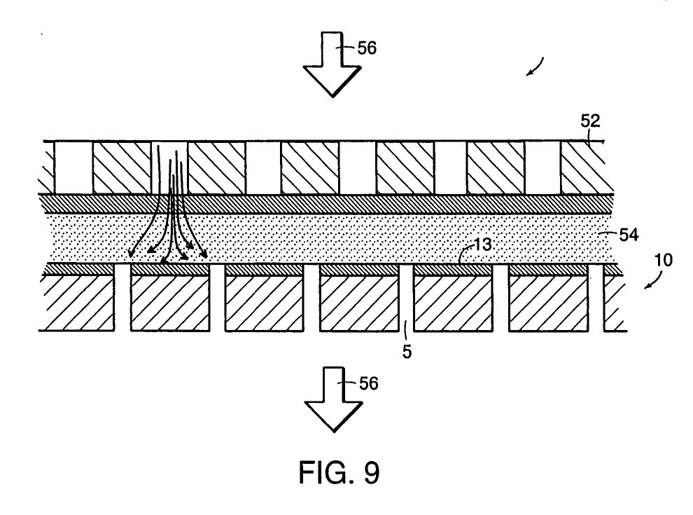

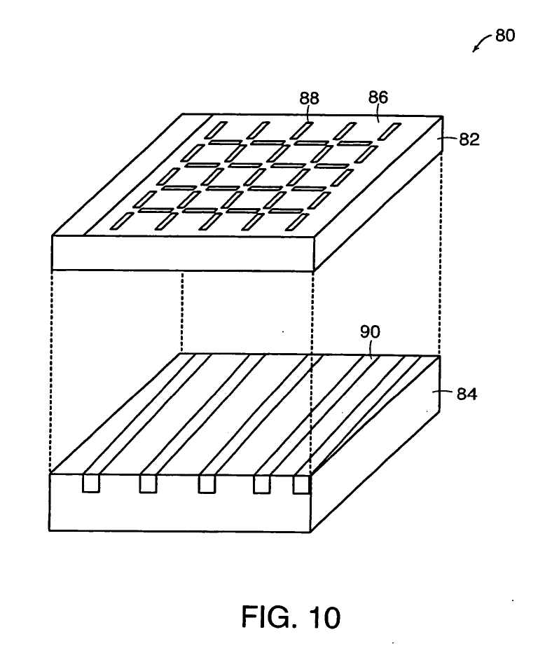

FIG. 9 illustrates elution of samples from a two dimensional gel 54 to the sample holder 10 of the present invention. A grid 52 supporting a two dimensional gel 54 comprising separated biopolymer molecules is disposed over a sample holder 10. A solution 56 passes through the grid 52 and the sample holder 10 to selectively elute and transfer separated biopolymer molecules onto support surfaces 13 of the sample holder 10. The solution 56 drains away from the sample holder 10 by passing through the sumps 12 and the holes 5.

Alternatively, a microscopic dot printer can load the samples onto the sample support surfaces 13. In one embodiment, the microscopic printing mechanism includes a robotically controlled dispenser moving from one sample support surface 13 to another surface 13 on the sample holder 10, depositing a desired sample at each sample support surface 13. For example, a dispenser may include a micropipette that contains a sample, and a robotic system that controls the position of the micropipette relative to the sample holder 10. The dispenser may include a series of micropipettes or an array of micropipettes to deliver multiple samples. In another embodiment, the dispenser comprises an ink jet printing device. Any appropriate means of loading a sample or samples onto the sample holder 10 that is known in the art or to be discovered in the art may be used in accordance with the present invention. U.S. Pat. No. 5,599,695, which describes a variety of microscopic printing mechanisms, is incorporated herein by reference.

In other embodiments, the sample holder 10 also may be used as a substrate in proteomic or genomic research The terms “proteomics” or “genomics”, as used herein refer to the science of proteins or DNAs to synthesize or to study multiple different substances more or less simultaneously. The sample holder 10 of the present invention permits selective analysis or synthesis of a sample of a multiplicity of samples placed on a single substrate. The sample holder 10 further permits repeat addressing of a sample disposed on a sample support surface 13 for multiple analysis.

In one embodiment, a two dimensional gel electrophoresis first separates biological polymers in a complex sample and the separated polymers are blotted onto the sample holder 10 of the present invention. Either before or after blotting, each polymer is digested to create multiple fragments of each biopolymer, e.g., a ladder sequence. The separated fragmented proteins or DNAs are then analyzed in one of numerous ways, such as mass spectrometry, to determine, for example, their sequence.

In another embodiment, the sample holder 10 of the present invention is used as a chemistry substrate for fabricating a library of polymers (e.g., PNAs), and more specifically, a library of biopolymers. The biopolymers, for example include but are not limited to, peptides, oligonucleotides, and organic molecules. A library of biopolymers, for example, may be fabricated by first placing a multiplicity of reactants on the sample support surfaces such that each reactant is disposed on one or a few adjacent sample support surfaces 13. The reactants then are exposed to different monomers, which potentially interacts with the reactants. The reactants, for example, may be linker molecules. In this manner, a different chemical reaction may be created at each sample support surface 13 at will. An array comprising a library of biopolymers can be used in an analysis process such as hybridization or drug screening. U.S. Pat. No. 5,605,662, which describes microelectronic systems for carrying out molecular biological reactions, such as nucleic acid hybridization, antibody/antigen reactions, clinical diagnostics and biopolymer synthesis, is incorporated herein by reference.

In still another embodiment, the sample holder 10 comprises libraries of unimolecular, double-stranded oligonucelotides formed on the sample support surfaces 13. The double-stranded oligonucleotides may be formed by first placing a first portion of an oligonucleotide on a sample support surface 13 and subsequently exposing the first portion to a second portion of the oligonucleotide. These libraries are useful in pharmaceutical discovery for the screening of numerous biological. samples for specific interactions between the double-stranded oligonucelotides and peptides, proteins, drugs and RNA U.S. Pat. No. 5,556,752, which describes fabrication of unimolecular-double-stranded oligonucleotides on a substrate, is incorporated herein by reference.

It is understood that the embodiments shown are exemplary and that it is intended to limit the scope of the invention only by the scope of the appended claims.

1. A sample holder comprising a substrate microfabricated to define
a multiplicity of microscopic islands defining sample support surfaces; and
at least one sump which separates adjacent said surfaces and inhibits transport of samples between adjacent said surfaces.

2-29. (canceled)
